(19) United States
(12) Patent Application Publication
(10) Pub. No.: US 2026/0028622 A1
(43) Pub. Date: Jan. 29, 2026

(54) RNA NANOSTRUCTURES WITH BASE MODIFICATIONS FOR REDUCING INNATE IMMUNOGENICITY

(71) Applicants: Hao Yan, Gilbert, AZ (US); Di Liu, Tempe, AZ (US); Liangxiao Chen, Tempe, AZ (US); Xinyi Tu, Tempe, AZ (US); Aleksandra Petrova, Tempe, AZ (US)

(72) Inventors: Hao Yan, Gilbert, AZ (US); Di Liu, Tempe, AZ (US); Liangxiao Chen, Tempe, AZ (US); Xinyi Tu, Tempe, AZ (US); Aleksandra Petrova, Tempe, AZ (US)

(73) Assignee: Arizona Board of Regents on behalf of Arizona State University, Scottsdale, AZ (US)

(21) Appl. No.: 19/282,949

(22) Filed: Jul. 28, 2025

Related U.S. Application Data
(60) Provisional application No. 63/676,077, filed on Jul. 26, 2024.

(51) Int. Cl. C12N 15/11; A61K 31/7115

(52) U.S. Cl. CPC C12N 15/11; A61K 31/7115; C12N 2310/333; C12N 2310/50

(57) ABSTRACT

RNA nanostructures with significantly reduced immunogenicity are described herein, enhancing their therapeutic utility. Incorporating specific base modifications during in vitro transcription decreases recognition by innate immune sensors. This breakthrough enables safer and more effective applications in targeted drug delivery and gene therapy, representing a significant advancement over current RNA-based technologies.

### CROSS-REFERENCE TO RELATED APPLICATIONS

This application is a non-provisional and claims benefit of U.S. Provisional Application No. 63/676,077 filed Jul. 26, 2024, the specification of which is incorporated herein in their entirety by reference.

### FIELD OF THE INVENTION

The present invention features RNA nanostructures with significantly reduced immunogenicity, enhancing their therapeutic utility. By incorporating specific base modifications during the in vitro transcription process, the resulting RNA nanostructures exhibit decreased recognition by the immune system's innate sensors.

### BACKGROUND OF THE INVENTION

Nucleic acid nanotechnology enables the precise assembly of nucleic acid molecules into nanostructures with defined sizes, shapes, and chemical compositions. These programmable nanostructures—synthesized through controlled processes and exhibiting excellent biocompatibility—have been applied across a range of biological functions, including modulation of protein translation, regulation of cell motility, and activation of immune responses. RNA nanotechnology, a rapidly developing subfield that combines the unique structural and functional properties of RNA with the design principles of nanotechnology, is now beginning to demonstrate significant promise in therapeutic applications. This growing significance is further highlighted by the recent successful development of mRNA vaccines against COVID-19, which has invigorated research into the utilization of RNA nanostructures for improved RNA-based therapeutics. However, it is imperative to acknowledge that the double-stranded RNA regions in these RNA nanostructures are intrinsically immunogenic, acting as innate immune stimulators that are recognized by the pattern recognition receptors (PRRs) including both endosomal (such as Toll-like receptor 3, TLR3) and cytosolic (such as retinoic acid-inducible gene I, RIG-I, and melanoma differentiation-associated protein 5, MDA5). The activation of innate immune response to RNA nanostructures could potentially compromise the therapeutic efficacy of certain RNA-based medicines, and, more severely, might even lead to cell apoptosis or necroptosis through the downstream pathways of PRRs.

Thus, the present invention provides compositions that mitigate the innate immunogenicity of RNA nanostructures by incorporating base modifications such as 5-methylcytosine (m5C), pseudouridine (ψ), and N1-methylpseudouridine (m1ψ).

### BRIEF SUMMARY OF THE INVENTION

It is an objective of the present invention to provide compositions and methods that allow for a reduction in innate immunogenicity as specified in the independent claims. Embodiments of the invention are given in the dependent claims. Embodiments of the present invention can be freely combined with each other if they are not mutually exclusive.

The effects of base modifications, including 5-methylcytosine (m5C), pseudouridine (ψ), and N1-methylpseudouridine (m1ψ), on the innate immunogenicity of RNA nanostructures were explored. The square-shaped single-stranded RNA origami (SQ) containing parallel crossover cohesion, which shows potent innate immune stimulatory effects both in vitro and in vivo, was employed as a case study. SQs with m5C and/or ψ/m1ψ modifications were successfully synthesized by in vitro transcription, and the shapes were not obviously altered by the modifications. While the cellular uptake of base-modified SQs remains largely unaffected, the innate immune responses in the mouse macrophage cell line Raw264.7 are reduced to different extents for both TLR3-mediated and RIG-I/MDA5-mediated immunities. Notably, incorporating both m5C and m1ψ into SQ almost completely eliminates the potent immune response of unmodified SQ. Although the underlying mechanisms are still being investigated, these findings open up promising opportunities for improving the safety profile of RNA nanostructure-enabled or -enhanced therapeutics.

In some embodiments, the present invention features an RNA nanostructure comprising a single RNA strand that includes a plurality of modified nucleotides, where the RNA strand is configured to self-assemble into the RNA nanostructure. In some embodiments, the single RNA strand forms parallel crossover cohesion interactions to create the RNA nanostructure. In some embodiments, the RNA nanostructure comprising a single RNA strand that includes a plurality of modified UTPs (e.g., pseudouridine (ψ), or N1-methylpseudouridine (m1ψ)) and modified CTPs (e.g., 5-methylcytosine (m5C)), where the RNA strand is configured to self-assemble into the RNA nanostructure.

In other embodiments, the present invention features a method of reducing innate immunogenicity in a subject in need thereof, the method comprising administering an RNA nanostructure as described herein to the subject.

One of the unique and inventive technical features of the present invention is the specific the utilization of base modifications (e.g., 5-methylcytosine (m5C), pseudouridine (4), and N1-methylpseudouridine (m1ψ)) applied during the in vitro transcription of RNA nanostructures. Without wishing to limit the invention to any theory or mechanism, it is believed that the technical feature of the present invention advantageously allows for a significantly reduction in the immunogenicity of the RNA nanostructures described herein. None of the presently known prior references or works have the unique inventive technical feature of the present invention.

Moreover, the prior references teach away from the present invention. For example, unlike previous approaches that primarily focused on mRNA for vaccines, the present invention expands the application to more complex RNA nanostructures used in broader therapeutic contexts. The process allows for the synthesis of RNA structures that retain their intended geometrical configurations while being less recognized by innate immune sensors, such as Toll-like receptors and cytoplasmic RNA sensors, thereby enhancing their biocompatibility and stability within biological systems. This differentiation offers substantial improvements over existing technologies by broadening the scope and effectiveness of RNA-based therapies.

Furthermore, the inventive technical feature of the present invention contributed to a surprising result. For example, the plurality of base modifications (e.g., 5-methylcytosine (m5C), pseudouridine (ψ), and N1-methylpseudouridine (m1ψ)) dramatically increased the melting temperature of the RNA nanostructures described herein. Compared with the non-modified versions, the dual-modification version of the RNA nanostructures has 15-degree higher melting temperature, indicating the resulting structure is more thermal stable.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee

The features and advantages of the present invention will become apparent from a consideration of the following detailed description presented in connection with the accompanying drawings in which:

FIGS. 1A and 1B show transcriptions-based strategy to integrate nucleoside analogues into ssRNA origamis. FIG. 1A shows non-limiting illustration of chemical structures of nucleoside analogues described herein, with four epigenetic nucleoside analogues and two therapeutic nucleoside analogues and FIG. 1B shows a schematic representation of nucleoside analogues integrated into the RNA Duplex of ssRNA Origami after transcription and annealing.

FIGS. 2A, 2B, 2C, 2D, 2E, and 2F show the characterization of nucleoside analogue-integrated ssRNA origamis. FIG. 2A shows a 1% agarose gel electrophoresis analysis of analogue-integrated ssRNA origamis after annealing. FIG. 2B shows an agarose gel electrophoresis analysis of 2000 nt ssRNA after transcription with varying stock ratios of dFdC using Y639F polymerase. FIG. 2C shows the atomic force microscopy images of ssRNA origami and its nucleoside analogue-integrated counterparts. Scale bar: 100 nm. FIG. 2D shows atomic force microscopy images of 50% and 20% integrated FUssROG. Scale bars: 100 nm. FIG. 2E shows ¹⁹F NMR reveals different loading ratios of FUssROG by adjusting the feedstock ratio of the FU nucleoside analogue. FIG. 2F NMR signal intensity of different FUssROG variants at −164.65 ppm.

FIGS. 3A, 3B, and 3C show efficient uptake of modified ssRNA origami by cells. FIG. 3A shows confocal microscopy images of Hela cells treated with Cy5-labeled SSRNA, Cy5-labeled ssRNA origami, and Cy5-labeled ssRNA origami in combination with an endocytosis inhibitor. Scale bar: 10 μm. FIG. 3B shows flow cytometry analysis of HeLa cells incubated with 5 μg/mL of Cy5-labeled ssRNA, Cy5-labeled ssRNA origami, and Cy5-labeled ssRNA origami in combination with 5 UM inhibitor for 8 h. FIG. 3C shows the mean intensity measurement derived from the flow cytometry analysis of Hela cells presented in FIG. 3B. The data are representative of at least three independent experiments

FIGS. 4A, 4B, 4C, 4D, and 4E show epigenetic nucleoside analogue-integrated ssRNA origami exhibits immune recognition regulatory functions. FIG. 4A shows a schematic illustration of epigenetic nucleoside analogue-integrated ssRNA origamis stimulate macrophages to exhibit varying degrees of immune activation after 24 h of incubation. FIG. 4B shows real-time quantitative reverse transcription PCR (RT-qPCR) analysis of innate immune-related gene expression of RAW 264.7 cells after incubation with nonmodified and PseudoU-integrated ssRNA origami. FIG. 4C shows flow cytometry data characterize the CD40 expression of macrophages stimulated by different epigenetic nucleoside analogue-integrated ssRNA origamis. FIG. 4D shows mean intensity measurement derived from the flow cytometry analysis of macrophages presented in FIG. 4C. FIG. 4E show flow cytometry analysis of CD40 and CD80 expression in macrophages following incubation with s2U-integrated ssRNA origami at varying loading ratios. The data are representative of at least three independent experiments.

FIGS. 5A, 5B, 5C, 5D, 5E, and 5F show therapeutic nucleoside analogue-integrated FUssROG promotes cancer cell autocrine production of type I interferons and potentiates chemotherapy. FIG. 5A shows a schematic illustration of the synergistic killing of cancer cells by FUssROG. FUssROG induces autocrine type I interferons in cancer cells, which leads to multiple antitumor responses and enhanced cytotoxicity. FIG. 5B shows agarose gel electrophoresis analysis of FUssRNA and FUssROG stability under different conditions. The samples were treated with 10% fetal bovine serum (FBS) in DMEM or 10 μg/mL RNase T2 in 10 mM MES buffer (pH=5.7) for 1 h. ImageJ was used to analyze the gray value of each lane. FIG. 5C shows RT-qPCR analysis of the immune relative mRNA expression levels in Patu 8988 cells after 24 h incubation with PBS, FU, ssRNA origami, and 50%/100% integrated FUssROG. FIG. 5D shows ELISA analysis of IFN-α level in Patu 8988 cell culture supernatant after 48 h treatment with PBS, FU, ssRNA origami, and 50%/100% integrated FUssROG. FIG. 5E shows mRNA expression level of Patu 8988 cell interferon-stimulated genes after the indicated treatment for 24 h. FIG. 5F shows cell viability of Patu 8988 cells after treatment with fluorouridine (7.5 μM), ssRNA origami (10 μg/mL), or FUssROG (10 μg/mL) for 72 h. The data were shown as a representative of at least three independent experiments and were analyzed using an unpaired t-test. *, **, *** and ns indicate p<0.05, p<0.01, p<0.001, and not significant, respectively.

FIGS. 6A, 6B, 6C, and 6D show atomic force microscopy analysis of ssRNA origami and FUssROG. FIGS. 6A and 6B show wide-field atomic force microscopy images of ssRNA origami and FUssROG respectively. Scale bar, 200 nm. FIGS. 6C and 6D show representative atomic force microscopy images of ssRNA origami and FUssROG (upright). The length and width of the origamis are indicated by black lines, respectively, in the lower panel. Scale bar, 50 nm.

FIGS. 7A, 7B, 7C, and 7D show serum cytokine levels in C57BL6 mice 3 hours post the intraperitoneal injection of 0.15 mg ssRNA origamis (non-modified, m5C-integrated and m5C/PseudoU-integrated). Data are shown as the mean±s.d. of n=3 biologically independent experiments.

FIGS. 8A and 8B show MIA-Paca2 cell response to FUssROG stimulation. FIG. 8A shows RT-qPCR of relative mRNA expression levels of MIA-Paca2 cells after 24-hour incubation with PBS, FU, ssRNA origami, and 50%/100%-integrated FUssROG. FIG. 8B shows ELISA analysis of IFN-α level in culture supernatant of Patu 8988 cell after 48-hour treatment with PBS, FU, ssRNA origami, and 50%/100%-integrated FUssROG.

FIGS. 9A, 9B, 9C, 9D, and 9E show cancer cell-autocrine type I interferons induce multiple anti-tumor responses and enhance cytotoxicity. FIG. 9A shows RT-qPCR of relative ISGs transcripts levels of MIA-Paca2 cells after 24-hour incubation with PBS, FU, ssRNA origami, and 50%/100%-integrated FUssROG. FIG. 9B shows RT-qPCR of relative ISGs transcripts levels of Hela cells after 24-hour incubation with PBS, FU, ssRNA origami, and 100%-integrated FUssROG. FIG. 9C-9E show cell viability of MIA Paca2 (FIG. 9C) and HeLa cell (FIG. 9D) after equivalent fluorouridine (7.5 μM or 3.75 μM), ssRNA origami (10 μg/mL), or 50%/100%-integrated FUssROG (10 μg/mL) treatment for 72 hours. The data were shown as a representative of at least three independent experiments and were analyzed using an unpaired t-test (FIG. 9C). The coefficient of drug interaction of ssRNA origami and FU is 0.18 for wild-type Hela cells and 1.77 for IFNAR1 K.D. Hela cells (FIG. 9E).

FIG. 10 shows the synthesis of ssRNA origami with different modifications. The atomic force microscopy characterization of the formation of ssRNA origami with or without base modifications. After the incorporation of ψ, m1ψ or m5C modification, the ssRNA origami can form the uniform shaped as the non-modified origami. The structure is still not influenced with the combination of ψ/m1ψ and m5C. The scale bar represents 200 nm.

FIGS. 11A and 11B show cellular uptake of ssRNA origami as observed by confocal microscopy (FIG. 11A) and flow cytometry (FIG. 11B). FIG. 11A shows immunofluorescence confocal microscopy of ssRNA origami in fixed Raw264.7 cells by anti-dsRNA monoclonal antibody J2 showing the cellular uptake of different origami. The cells were incubated with 5 μg/mL different origami for 4 hours and followed by intracellular IF staining of dsRNA with J2 monoclonal antibody as primary antibody and anti-mouse-AF488 antibody as secondary antibody. The nuclei were stained by Hoechst 33342 as counter stain. The scale bar represents 50 μm. Base-modifications did not inhibit the ssRNA origami uptake efficiency in macrophage. FIG. 11B (left panel) shows representative histogram of ssRNA origami uptake flow cytometry in Raw264.7 cells. The cells were incubated with 5 μg/mL different origami for 4 hours and followed by intracellular staining. The PBS treated group was used as a negative control for gating. The AF488 positive population was gated out, indicating the cellular uptake of ssRNA origami of different modifications. The right panel showed the flow cytometry quantitative analysis for ssRNA origami uptake in Raw264.7 cell. Data are shown as the mean±s.e.m. of biological replicates (n=3 per group). Base-modifications did not inhibit the ssRNA origami uptake efficiency in macrophage.

FIGS. 12A-12C show the innate immune responses induced by ssRNA origami with different base modifications in various macrophage reporter cell lines. FIG. 12A shows interferon (IFN) activity in Raw-Lucia-ISG, Raw-MAVS-KO-Lucia-ISG, and Raw-TRIF-KO-Lucia-ISG cell lines. Cells were incubated with 5 g/mL ssRNA origami containing various modifications or PBS control for 24 hours. IFN activation was measured by luciferase relative luminance units (RLU). Data are presented as mean±s.e.m. (n=3 biological replicates per group). The results show that pseudouridine (ψ) and N1-methylpseudouridine (m1ψ), particularly in combination with 5-methylcytosine (m5C), reduce the innate immune response, with m1ψ+m5C effectively eliminating IFN activation FIG. 12B depicts NF-κB activity assessed by CD40 expression using flow cytometry. The same cell lines were treated as described above, followed by staining with CD40-PE antibody. CD40 mean fluorescent intensity (MFI) reflects NF-κB activity, and results are shown as mean±s.e.m. (n=3). The data indicate that base modifications suppress the TLR3-TRIF-mediated NF-κB response. FIG. 12C shows the innate immune response triggered by cytosolic receptor pathways. Cells were transfected with 1 μg/mL modified ssRNA origami, Lipofectamine control, or PBS control for 24 hours. IFN activity was measured via luciferase RLU, normalized to CCK-8-based relative cell viability. Data are shown as mean±s.e.m. (n=3). Results confirm that base modifications also suppress cytosolic receptor-mediated immune responses. Bars from left to right represent no modification, ψ, m1ψ, m5C, ψ and m5C, m1ψ and m5C, and PBS.

### DETAILED DESCRIPTION OF THE INVENTION

Disclosed are various peptides, solvents, solutions, carriers, and/or components to be used to prepare compositions to be used within the methods disclosed herein. Also disclosed are the various steps, elements, amounts, routes of administration, symptoms, and/or treatments that are used or observed when performing the disclosed methods, as well as the methods themselves. These and other materials, steps, and/or elements are disclosed herein, and it is understood that when combinations, subsets, interactions, groups, etc. of these materials are disclosed, that while specific reference of each various individual and collective combination and permutation of these compounds may not be explicitly disclosed, each is specifically contemplated and described herein. It is also to be understood that the terminology used herein is for the purpose of describing particular embodiments only and is not intended to be limiting.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessarily solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

All publications, patent applications, patents, and other references mentioned herein are incorporated by reference in their entirety for all purposes. In case of conflict, the present specification, including explanations of terms, will control.

Although methods and materials similar to or equivalent to those described herein can be used to practice or test the disclosed technology, suitable methods and materials are described below. The materials, methods, and examples are illustrative only and not intended to be limiting.

As used herein, the term “single-stranded RNA (ssRNA)” refers to an RNA nanostructure formed from a single RNA strand.

Referring now to the figures, in some embodiments, the present invention features RNA nanostructures with significantly reduced immunogenicity and enhanced therapeutic utility by incorporating specific base modifications during in vitro transcription, resulting in decreased recognition by the immune system's innate sensors. For example, the present invention may feature an RNA nanostructure comprising a single RNA strand that includes a plurality of modified NTPs, e.g., modified ATPs, modified CTPs, modified UTPs, modified GTPs, or a combination thereof.

Without wishing to limit the present invention to any theory or mechanism, it is believed that single-stranded RNA nanostructure (e.g., RNA origami nanostructures) without base modifications triggers strong innate immune response through cytosolic dsRNA sensors RIG-I/MDA5 or endosomal TLR3. For example, the cytosolic signal is transduced to mitochondrial antiviral signaling (MAVS) while the endosomal signal from TLR3 is transduced to the TIR domain containing adaptor molecule 1 (TRIF). Then both signaling pathways can mediate NF-κB and IRFs activation for innate immunity. When the m5C, ψ and m1ψ were incorporated into the single-stranded RNA origami, the innate immune responses are be suppressed to different extent. The innate immune response is found to be eliminated by a combination of m5C and m1ψ modifications.

The present invention may feature an RNA nanostructure comprising a single RNA strand that includes a plurality of modified nucleotides. The RNA strand is capable of self-assembling into the RNA nanostructure. In some embodiments, the single RNA strand forms parallel crossover cohesion interactions that promote or stabilize formation of the nanostructure. In some embodiments, the modified nucleotides comprise one or more modified nucleoside triphosphates including, for example, modified uridine triphosphates (UTPs), modified cytidine triphosphates (CTPs), modified adenosine triphosphates (ATPs), modified guanosine triphosphates (GTPs), or any combination thereof.

In certain embodiments, the RNA nanostructure comprises a single RNA strand that includes a plurality of modified uridine triphosphates (UTPs), modified cytidine triphosphates (CTPs), or a combination thereof. The modified RNA strand is capable of self-assembling into the RNA nanostructure.

In some embodiments, the present invention features a RNA nanostructure (e.g., a ssRNA nanostructure) comprising a single RNA strand that includes a plurality of modified UTPs and CTPs. In some embodiments, the modified RNA strand is capable of self-assembling into the RNA nanostructure. For example, the single RNA strand may form parallel crossover cohesion interactions that promote or stabilize formation of the nanostructure.

In some embodiments, the present invention features a single-stranded RNA (ssRNA) nanostructure comprising a single RNA strand that includes a plurality of modified nucleotides. The modified nucleotides may include, for example, modified uridine triphosphates (UTPs), modified cytidine triphosphates (CTPs), modified adenosine triphosphates (ATPs), or modified guanosine triphosphates (GTPs), either individually or in combination.

Non-limiting examples of modified UTPs include but are not limited to pseudouridine (ψ), N1-methylpseudouridine (m1ψ), 2-thiouridine (s2U), 5-methyluridine, or fluorouracil (5FU). Non-limiting examples of modified CTPs include but are not limited to 5-methycytosine (m5C), N4-acetylcytosine (ac4C), 5-Hydroxymethylcytidine (5hmC), or 2′,2′-diflurodeoxycytidine (dFdC).

In certain embodiments, the present invention features an RNA nanostructure (e.g., a ssRNA nanostructure) comprising a single RNA strand that includes a plurality of 5-Methylcytosine (5mC) and N1-methylpseudouridine (m1ψ). In some embodiments, the single RNA strands forms parallel crossover cohesion interactions to create the RNA nanostructure. Without wishing to limit the present invention to any theory or mechanism it is believed that the synergistic effect of 5-methylcytosine (5mC) and N1-methylpseudouridine (m1ψ) to diminish the immune response to levels comparable with negative controls.

Non-limiting examples of modified ATPs may include but are not limited to N1-methyladenosine (m1A), N6-methyladenosine (m6A), or inosine (I), N6,2′-O-dimethyladenosine (m6Am). Non-limiting examples of modified GTPs may include, but are not limited to, 7-methylguanosine (m7G), or 8-oxo-7,8-dihydroguanosine (8-oxoG).

In some embodiments, the RNA nanostructures may be assembled and folded from more than two strands of RNA. For example, the RNA nanostructure may comprise one or more strands of RNA. In some embodiments, the RNA nanostructures comprise two or more strands of RNA.

In some embodiments, the RNA is selected from the group consisting of messenger RNA (mRNA), transfer RNA (tRNA), ribosomal RNA (rRNA), small interfering RNA (siRNA), microRNA (miRNA), and synthetic or engineered RNA analogs thereof.

In some embodiments, the RNA nanostructures described herein may be formed as a domain of a larger RNA, such as mRNAs or non-coding RNAs.

In some embodiments, the nanostructure is rectangular in shape. The nanostructures described herein may also be in the shape of a rhombus, parallelogram, heart, triangle, square, ladder, or polyhedron.

In other embodiments, the present invention features an innovative approach for synthesizing RNA nanostructures with reduced immunogenicity by incorporating base modifications during in vitro transcription. In some embodiments, the modified nucleoside triphosphates (NTPs) are mixed with standard NTPs to create modified RNA, which is then structured into a square shape using annealing processes. Atomic force microscopy (AFM) evaluates the structural formation, while analyses of melting temperature and biostability demonstrate the modifications' effects. The immunogenicity of modified ssRNAOG is then evaluated by applying them to a macrophage cell line featuring an immune response reporter luciferase gene. Techniques such as reverse transcription quantitative PCR (RTq-PCR) ascertain cellular uptake, whereas incubation and transfection experiments test the activation of TLR3 and RIG-I receptors, key to the immune response. The assessment uses flow cytometry and specific protein knockout cell lines to quantify this response accurately.

In some embodiments, the compositions described herein reduce innate immunogenicity. Innate immunogenicity may be reduced by decreasing the response of a TLR signaling pathway or a response of an RIG-I signaling pathway.

The present invention may also feature a method of reducing innate immunogenicity in a cell. In some embodiments, the method comprising contacting the cell with an RNA nanostructure (e.g., a ssRNA nanostructure) as described herein. For example, an RNA nanostructure comprising a single RNA strand that includes a plurality of modified nucleotides, where the RNA strand is configured to self-assemble into the RNA nanostructure. In some embodiments, contacting the cell comprises transfecting the cell with the RNA nanostructure or direct incubation of the cells with the RNA nanostructure.

The present invention may further feature a method of reducing innate immunogenicity in a subject in need thereof. In some embodiments, the method comprise administering an RNA nanostructure (e.g., a ssRNA nanostructure) as described herein to the subject. For example, an RNA nanostructure comprising a single RNA strand that includes a plurality of modified nucleotides, where the RNA strand is configured to self-assemble into the RNA nanostructure.

In some embodiments, the present invention features a method of treating cancer in a subject in need thereof. In some embodiments, the method comprise administering an RNA nanostructure (e.g., a ssRNA nanostructure) as described herein to the subject. For example, an RNA nanostructure comprising a single RNA strand that includes a plurality of modified nucleotides, where the RNA strand is configured to self-assemble into the RNA nanostructure.

Other types of RNA nanostructures may be used in accordance with the present invention, including but not limited to RNA tiles or ssRNA origami based on kissing loops.

### Example

The following is a non-limiting example of the present invention. It is to be understood that said example is not intended to limit the present invention in any way. Equivalents or substitutes are within the scope of the present invention.

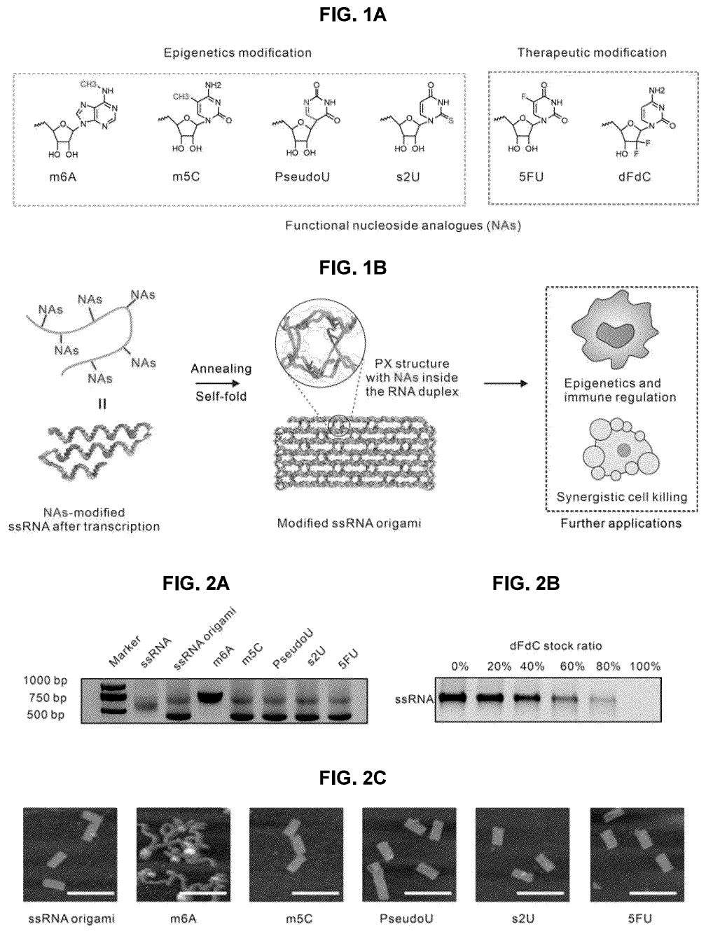

Multiple nucleoside analogues were controllably incorporated into a 2000 nucleotide (nt) ssRNA origami using transcription methods, which resulted in a modified ssRNA origami with enhanced biomedical functions (FIGS. 1A and 1B). Most functional nucleoside analogues could be controllably integrated into ssRNA origamis while maintaining the structural properties of the origami. After the integration of epigenetic modification bases, the ssRNA origamis exhibited tunable immune recognition and regulatory properties. Moreover, the ssRNA origamis integrated with therapeutic bases demonstrated sequential release characteristics, thereby inducing cell self-secretion of type I interferons and gradually releasing chemotherapeutic drugs, which ultimately enhanced cancer cell killing. The present invention features a novel and quantitative approach for integrating “molecular” modules into ssRNA origami, thereby further enriching their biomedical functions.

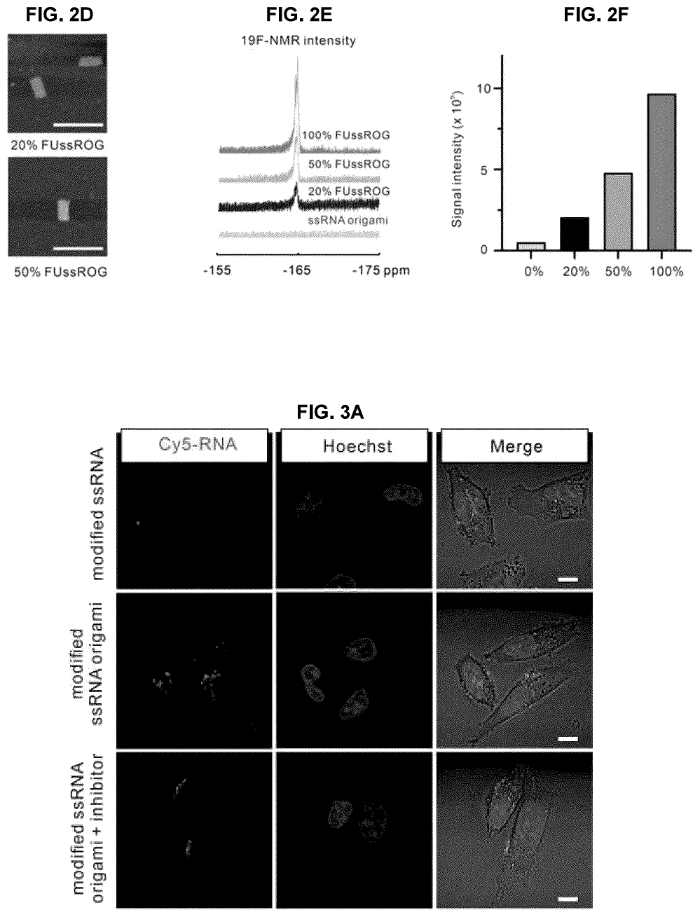

The synthesis process is illustrated in FIGS. 1A and 1B, where functional nucleoside analogues in the form of triphosphates (NTPs) were precisely added to the transcription system to transcribe into a 2000 nt ssRNA. Subsequently, through annealing, the nucleoside analogues-integrated ssRNA strands self-folded into a rectangular ssRNA origami, which consists of knot-free paranemic crossover (PX) motifs. Specifically, as an example, FIGS. 1A and 1B shows the integration of four common epigenetic nucleoside analogues (m6A, m5C, PseudoU, s2U) into ssRNA origami. After the transcription and annealing processes, the folding condition of the synthesized ssRNA origami was evaluated by using agarose gel electrophoresis (FIG. 2A). The agarose gel plots revealed that unmodified ssRNA origami exhibited migration rates similar to those of the m5C-, s2U-, and PseudoU-integrated ssRNA folded products. However, the m6A-modified folded products exhibited slower migration and may indicate a lack of proper folding, which could be further supported by the scattered m6A RNA structures in the AFM imaging in FIG. 2C. This failure in folding for the m6A modification can be attributed to the methyl group, which induced a rotation of the carbon-nitrogen bond and resulted in the display of the methyl group in the anti-conformation. This conformational alteration destabilized the RNA duplex during base-pairing with U, thereby hindering the successful folding of the ssRNA origami.

Next, therapeutic nucleoside analogues of dFdC (a cytidine analogue) were integrated into a 2000 nt ssRNA. However, as the dFdC ratio increased during transcription, a significant decrease in RNA yield was observed, which eventually lead to its complete disappearance, as depicted in FIG. 2C. To address this issue, dFdC was integrated into a shorter 99 nt ssRNA and achieved an acceptable yield. Nonetheless, polyacrylamide gel electrophoresis (PAGE) analysis revealed difficulties in folding the dFdC-integrated ssRNA into a paranemic crossover structure, as evidenced by the indistinguishable migration rates before and after annealing of the dFdC-integrated ssRNA. This may be attributed to the substitution of two fluorine atoms on the sugar ring of cytidine in dFdC, thereby impacting the recognition by RNA polymerase during transcription and the complementary pairing necessary for structural folding. Subsequently, an alternative therapeutic nucleoside analogue of fluorouridine was tested, which incorporates a single-atom modification by replacing a hydrogen atom with a fluorine atom in uridine. Triphosphorylated fluorouracil (FU) proved to be efficiently transcribed and integrated into ssRNA origami (FIG. 2A, last lane). These findings validate the successful integration of most functional nucleoside analogues into ssRNA origamis.

The influence of integrating nucleoside analogues on the morphology of ssRNA origami was then investigated. Atomic force microscopy (AFM) analysis revealed that both the non-modified and nucleoside analogue-integrated ssRNA origamis exhibited a single-layer rectangular structure with no discernible morphological changes, except for the m6A-integrated ssRNA, which failed to fold into the correct origami structure (FIG. 2C). Further examination of the nanostructure demonstrated that the length (˜40 nm), width (˜19 nm), and height (˜2 nm) of the ssRNA origami remained unaltered in the presence of the modified bases (FIGS. 6A-6D). Thus, the integration of nucleoside analogues of m5C, PseudoU, s2U, and FU had minimal impact on the overall structure of ssRNA origami.

The quantitative integration of nucleoside analogues into ssRNA origamis was further validated by utilizing fluorouridine (FU) as an illustrative example because of its distinct NMR signals facilitated by the fluorine atom. By controlling the stock ratio of FU nucleoside analogues from 100% to 20%, no discernible morphological changes in the FU-integrated ssRNA origami (FUssROG), regardless of the loading ratios of FU (FIG. 2D). The ¹⁹F NMR results indicated a positive correlation between the loading ratio of FU in the FUssROG and the stock ratio employed (FIGS. 2E and 2F). These findings highlight the ability to modulate the stock ratio of epigenetic nucleoside analogues to achieve corresponding loading ratios in RNA molecules. Overall, this result demonstrates that we can controllably modify the basis of ssRNA origami through a transcription-based strategy.

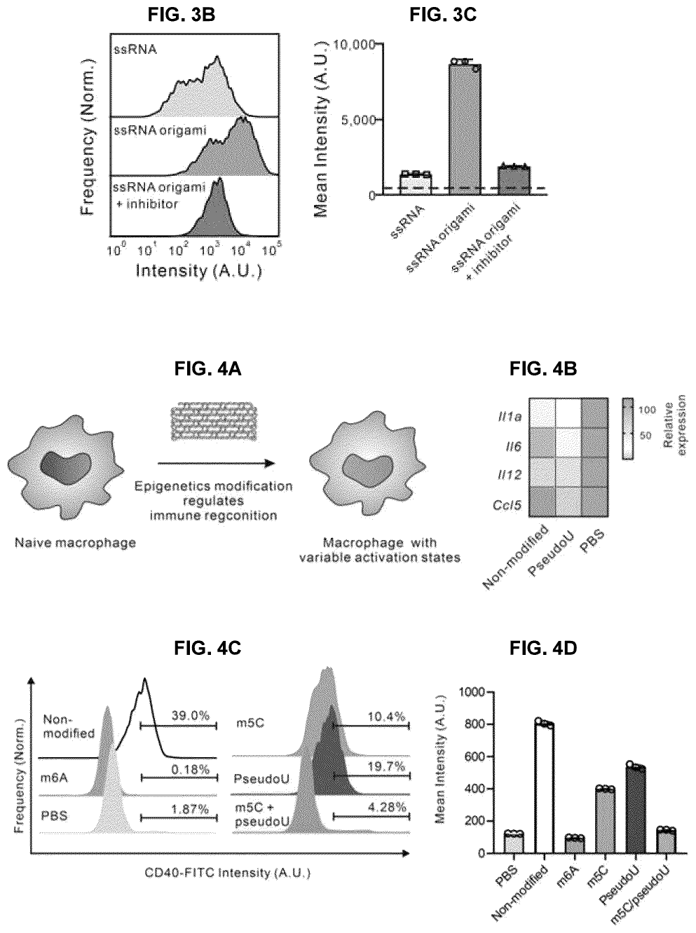

To verify the cell uptake of the modified ssRNA origami, confocal fluorescence microscopy and flow cytometry was used to characterize the uptake of Cy5-labeled ssRNA origami by cancer cells. As shown in FIG. 3A, confocal imaging results revealed that ssRNA origami was more localized within cells compared with Cy5-labeled ssRNA, thereby indicating a preferential cell uptake of structured ssRNA origami for epigenetic and therapeutic nucleoside analogues. This observation was further supported by flow cytometry experiments, where the mean fluorescence intensity of cancer cells treated with Cy5-labeled ssRNA origami was approximately six times higher than that of the ssRNA control group (FIGS. 3B and 3C). Since efficient delivery can exert a greater impact on cells, the integration of epigenetic and therapeutic nucleoside analogues into ssRNA origami may result in enhanced bioeffects. Furthermore, the addition of the endocytosis inhibitor prochlorperazine significantly reduced the uptake of modified ssRNA origami by cancer cells, thereby demonstrating that the endocytosis pathway was indispensable for the cellular uptake of modified ssRNA origami. Thus, structured ssRNA origami enhances the endocytic uptake by cells, which could provide a basis for subsequent cellular applications.

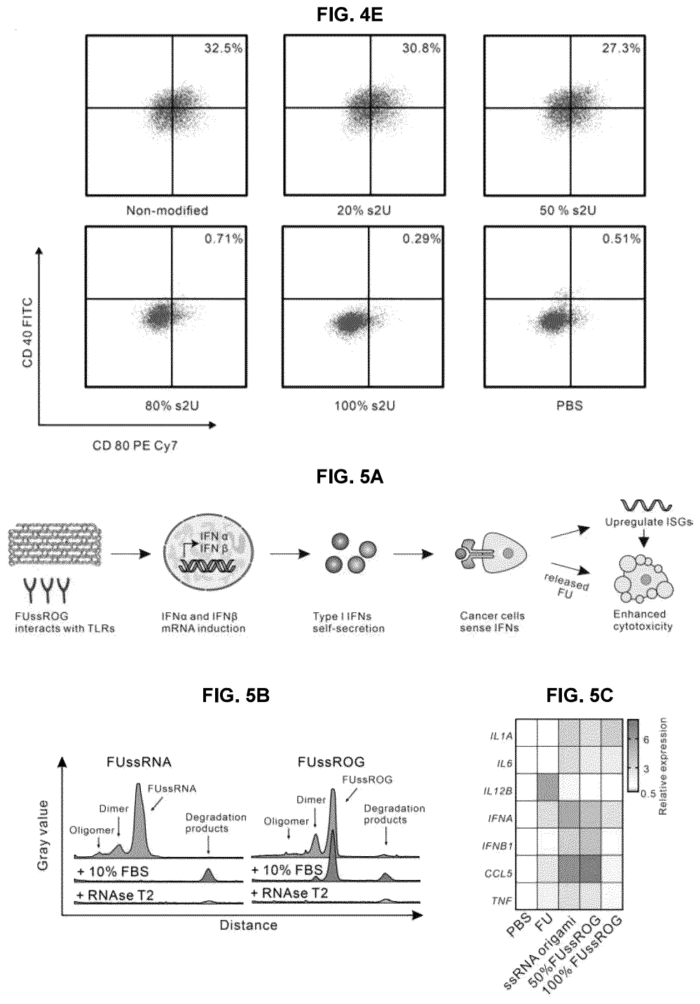

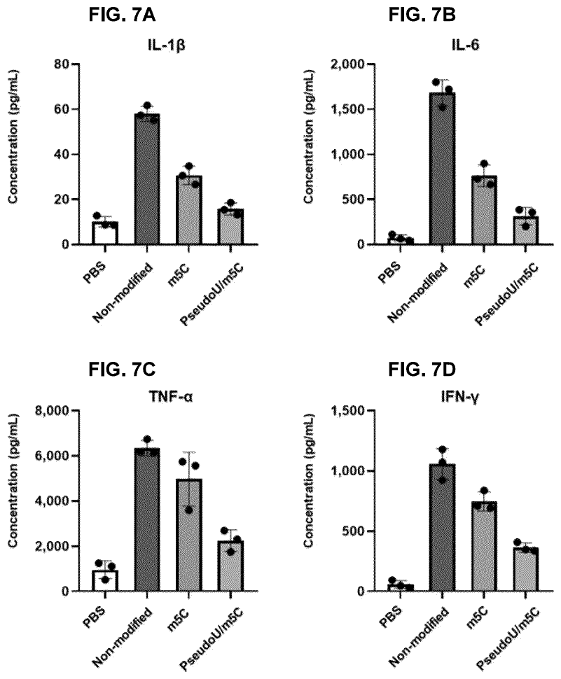

The potential for epigenetic analogues to alter the innate immunostimulatory properties of ssRNA origami was investigated. To evaluate the immune stimulation effects of modified ssRNA origami structures, mouse macrophage RAW 264.7 cells were utilized as a reporter cell line. At the mRNA level, modification of ssRNA origami with PseudoU led to a downregulation of II1a, II6, II12, and Ccl5 transcripts compared with the unmodified ones, thereby indicating a modulated immune activation function. The immune activation function of epigenetically integrated ssRNA origami was further verified with CD40 and CD80 markers. Flow cytometry results showed that unmodified ssRNA origami strongly activated macrophages, with an activation rate of 39% (FIG. 4C). Interestingly, the m6A-integrated ssRNA structure exhibited negligible immune activation ability comparable with that of the blank control (PBS), with an activation rate of less than 1%. Two reasons may collaboratively attribute to this phenomenon: (1) m6A modification disrupts dsRNA binding to TLR3, thereby enabling RNA immune suppression, and (2) insufficient folding may impact the cell uptake of the m6A-integrated ssRNA structure, thereby leading to decreased effective concentration in endolysosomes. Furthermore, the impact of single modifications of m5C and PseudoU was investigated on macrophage activation, which resulted in modest reductions in the activation rate to 19% and 10%, respectively. Notably, the incorporation of both nucleoside analogues into the RNA origami structure significantly decreased macrophage activation to 4%. The nucleoside modification of RNA is related to immune recognition and translation efficacy, which significantly affects the effect of mRNA in vivo. Following intraperitoneal administration of ssRNA origami in mice, m5C modification slightly reduced the release of proinflammatory cytokines (IL-1β, IL-6, TNF-α, and IFN-γ) in serum compared with unmodified origami, while the dual modification of m5C and PseudoU significantly suppressed cytokine production (FIGS. 7A-7D). These results together confirmed that epigenetic modifications in ssRNA origami significantly influence their immune recognition function both in vitro and in vivo.

The impact of quantitative modifications was further investigated on immune recognition using s2U as an example. By controlling the integration ratio of s2U, 20% and 50% s2U-integrated ssRNA origami can still activate the immune system, while 80% and 100% s2U-integrated ssRNA origami essentially evaded immune recognition. Overall, integrating epigenetics nucleoside analogues into ssRNA origami can alter the immunogenicity of the unmodified counterparts and confer epigenetic immune regulatory functions.

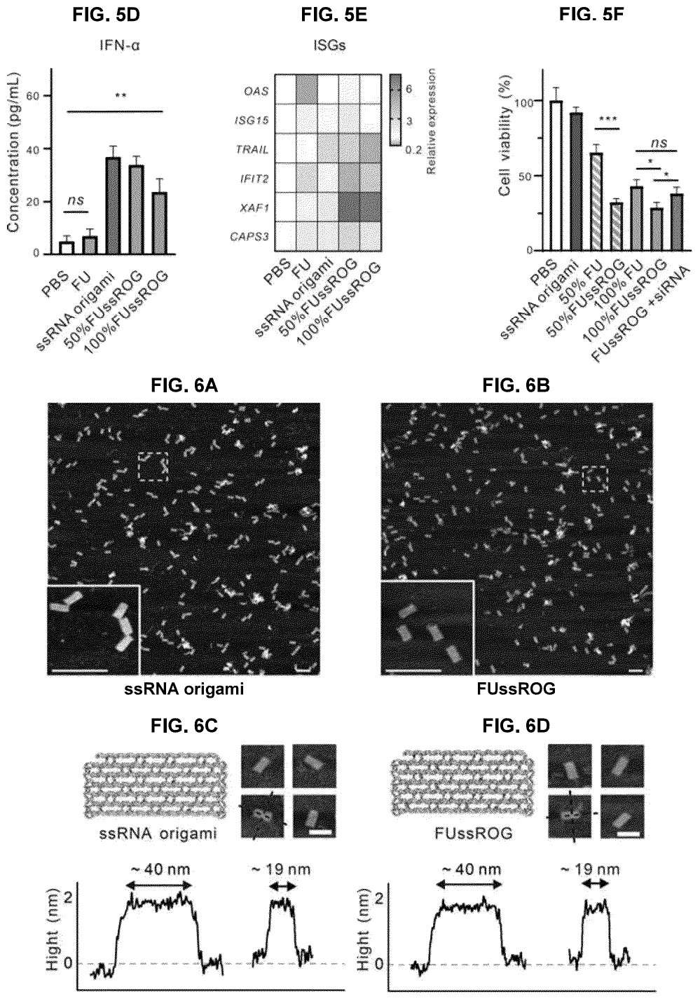

Considering the potential to regulate the immune activation capabilities of ssRNA origami through the integration of nucleoside analogues, the feasibility of achieving a synergistic approach combining chemotherapy and immunotherapy was subsequently explored by integrating therapeutic nucleosides into ssRNA origami. This integration allows for the safe control of immunogenicity and concurrent delivery of chemotherapeutic agents (FIG. 5A). The efficacy of drug release from FUssROG was first evaluated. Both precise intracellular drug release and the synchronous effect of combinatorial drugs in a temporally and spatially ordered manner are crucial for ensuring the safety and effectiveness of immunotherapy. After uptake by the cancer cell, the RNA nanostructure can be transported to the endolysosome, where highly active RNases, such as RNase T2, efficiently digest RNA. As shown in FIG. 5B, FUssRNA was completely degraded in 10% FBS after 1 h, whereas FUssROG remained stable in 10% FBS and only degraded after incubation with RNase T2 at a high concentration (10 μg/mL). This result indicates the biostability of FU-integrated ssRNA origami under physiological conditions and the efficient drug release in endolysosomes. Taken together, the structured FUssROG enables efficient drug delivery and controlled intracellular release in contrast to the loose single strands, thereby establishing a foundation for the concurrent utilization of its chemotherapeutic and immune activation functions.

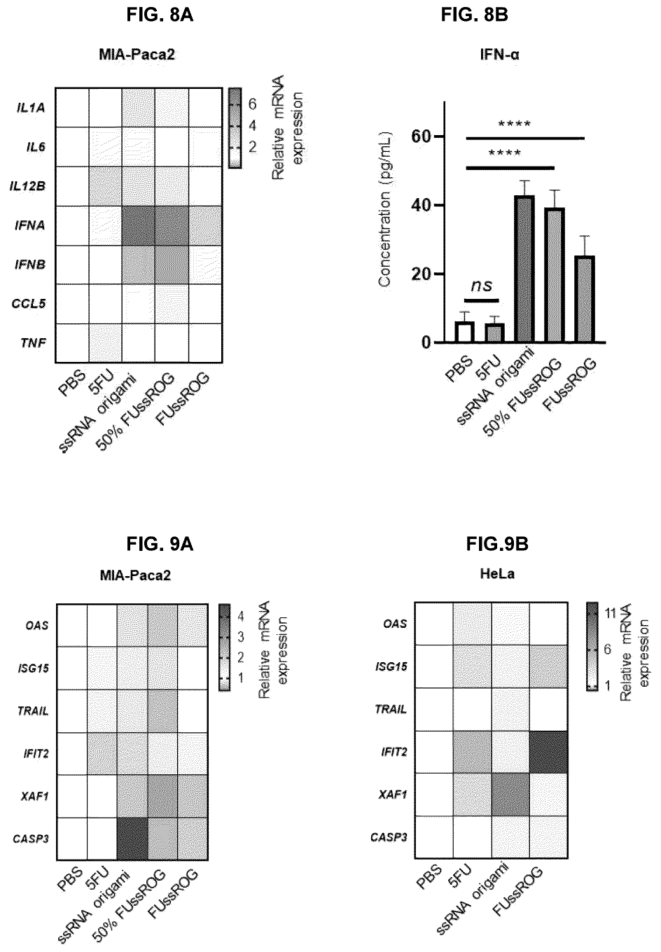

The self-secretion of interferon in cancer cells after chemotherapy is often associated with a positive therapeutic response, but not all chemo-drugs can directly induce interferon secretion. For instance, FU requires the induction of cell apoptosis and the release of damage-associated molecules before further activating the STING pathway to achieve interferon release, which limits its clinical indications and usage. Here, whether FUssROG could directly promote interferon secretion in cancer cells was investigated (FIG. 5A) to present enhanced therapeutic effects. RT-qPCR analysis demonstrated that both nonmodified ssRNA origami and FUssROG upregulated the expression of innate immune activation-related genes, including IL1A, IL6, and IL12B, as well as interferon genes in human pancreatic Patu 8988 cancer cells (FIG. 5C). In contrast, FU alone does not enhance the expression of interferon genes in pancreatic cancer cells. This was further supported by enzyme-linked immunosorbent assay (ELISA) experiments, which showed that an equivalent dose of FUssROG effectively promoted the self-secretion of interferon-alpha in cancer cells compared with FU (FIG. 5D). Similar results were observed in other human cancer cells, such as MIA PaCa-2 cells (FIG. 8A-8B). By controlling the integration ratio of FU, FU-integrated ssRNA origami leads to less upregulation of most innate immune activation-related genes than nonmodified ssRNA origami, while the 100%-integrated FUssROG induced lower gene expression than the 50%-integrated FUssROG (FIGS. 5C and 5D), suggesting that the immune response can be modulated by adjusting the stock ratios of FU. These findings indicate that ssRNA origami can serve as a controllable immunogenic drug delivery platform, thereby enhancing the innate immune activation effect of chemotherapy drugs and promoting interferon release.

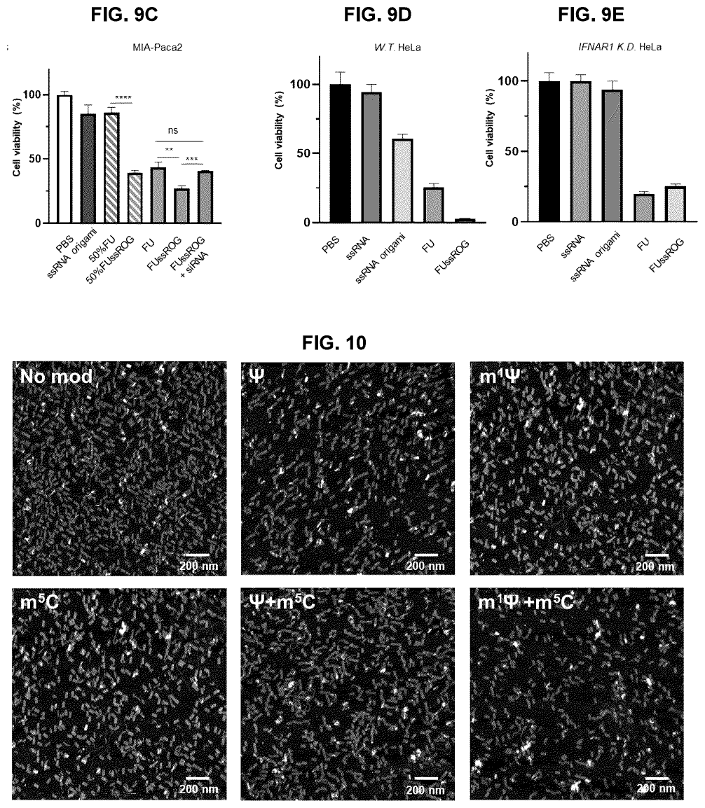

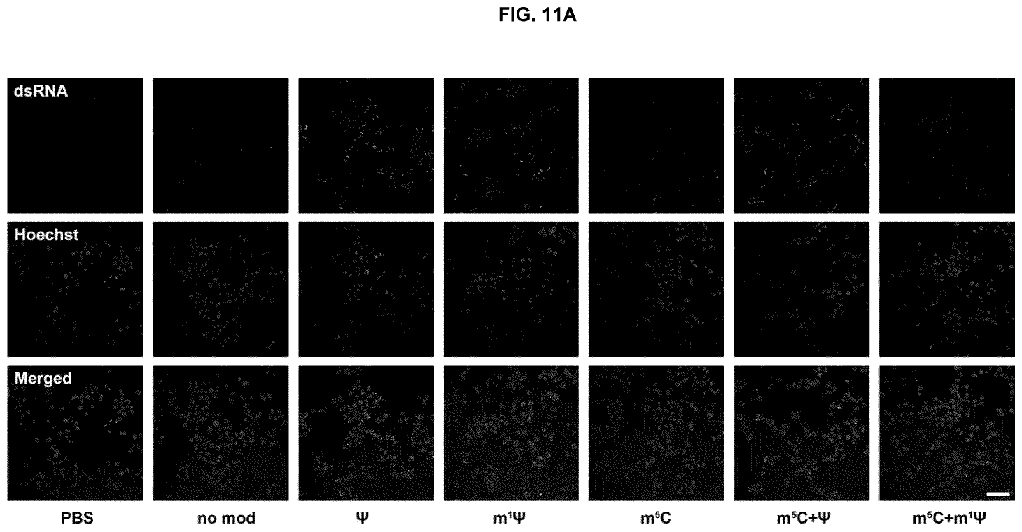

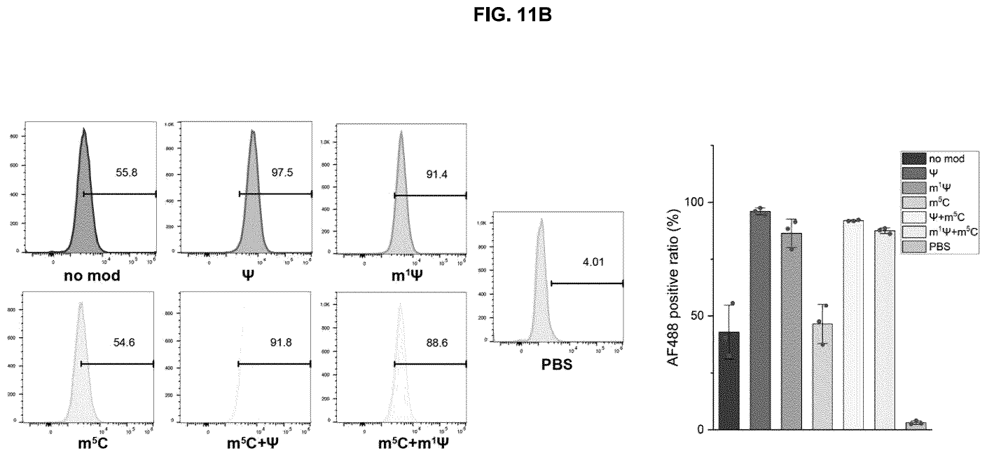

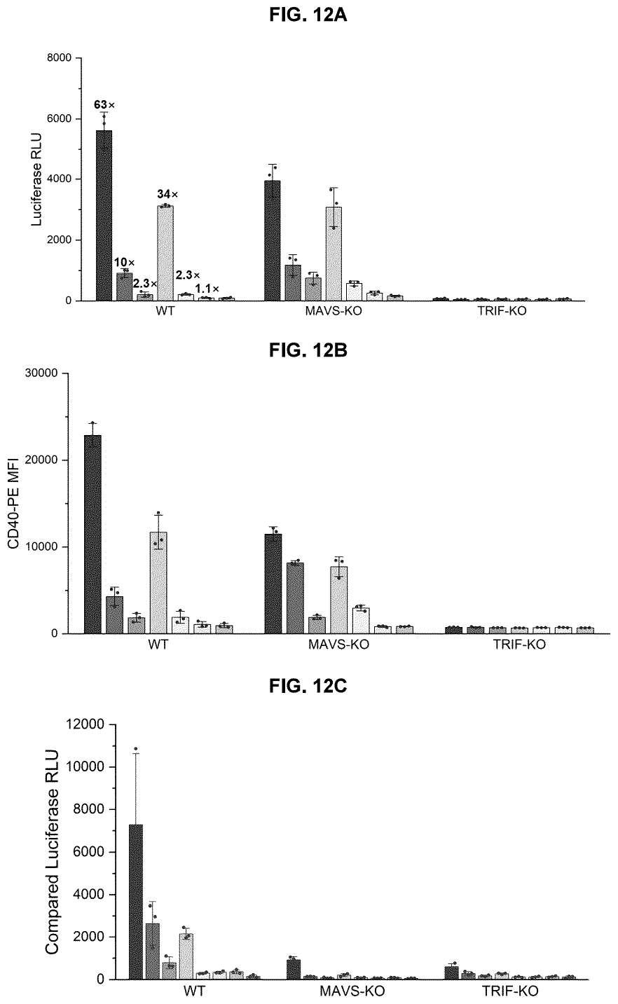

To evaluate cancer cell responses to interferons induced by FUssROG, the expression of interferon-stimulated genes (ISGs) was examined. As shown in FIG. 5E, both ssRNA origami and FUssROG can upregulate the expression of ISGs such as OAS, ISG15, TRAIL, and XAF1 in Patu 8988 cancer cells. These genes are known to potently and directly mediate apoptosis of cancer cells and sensitize cell response to chemotherapy. Similar results were also observed in MIA-Paca 2 cells and Hela cells (FIG. 9A-9B). Next, the cytotoxicity of FUssROG (FIG. 5F) was evaluated. While nonmodified ssRNA origami exhibited the lowest level of cytotoxicity, it induced the highest rate of self-secreted interferon. In contrast, when FU was integrated into ssRNA origami, the 50%/100% integrated FUssROG demonstrated greater effectiveness in killing Patu 8988 cancer cells compared with an equivalent dose of FU, alone. Notably, the coefficient of drug interaction is 0.5 for 50% integrated FUssROG and 0.7 for 100% integrated FUssROG, which indicates that a stronger synergistic effect of interferon therapy and chemotherapy can be achieved by 50% integrated FUssROG. An optimized integration ratio could potentially achieve a better therapeutic effect by balancing direct FU cytotoxicity and immune stimulation. Similar results were observed for MIA Paca-2 and HeLa cells (FIG. 9A-9E), thereby suggesting that integrating FU to ssRNA origami can serve as a broad-spectrum synergistic strategy to enhance cell cytotoxicity. Furthermore, efficient knockdown of the type I IFN receptor IFNAR1 using small interfering RNA (FIG. 5F) subsequently impaired the therapeutic effect of 100% integrated FUssROG on Patu 8988 cells (p=0.0405) and MIA Paca-2 cells (p=0.0003), which highlights the essential role of the autocrine interferon induced by FUssROG in enhancing cell-killing effects. In conclusion, these results underscore the successful synergy between chemotherapy and immunotherapy achieved through the integration of therapeutic nucleoside analogues into ssRNA origami.

In summary, the versatility of ssRNA origami as a platform for integrating functional nucleoside analogues at the molecular level to create nanostructures with expanded biomedical properties is demonstrated herein. Using transcription methods, therapeutic nucleoside analogues were efficiently and controllably integrated onto ssRNA origami, thereby resulting in controllably loaded and uniformly shaped nucleic acid nanostructures. These integrated ssRNA origami structures were effectively taken up by cells and subsequently digested to release the active agents, thereby facilitating their biomedical effects. Epigenetic nucleoside analogues endowed the ssRNA origami with immune regulatory functions, while therapeutic nucleoside analogues enhanced the synergistic killing effect of ssRNA origami on tumor cells.

Thus, the present invention provides a streamlined approach for developing multifunctional nucleic acid nanostructures at the molecular level using minimal components. Featuring tunable drug loading capacity, straightforward synthesis, and precise structural control, the integration of nucleoside analogues into ssRNA origami expands its functional capabilities beyond epigenetic modulation and interferon-based chemotherapy. Accordingly, the present invention is not limited to these applications and may encompass additional therapeutic or diagnostic uses.

Synthesis and characterization of modified and unmodified ssRNA origamis: FUTP can be chemically synthesized by tri-phosphorylation of FU. Briefly, FU (0.25 mmol) was first dissolved in trimethyl phosphate and cooled with an ice bath. POCl3 (0.5 mmol, 2.0 eq.) was added dropwise to the mixture, and stirring was continued for 18 hours on ice. Next, tributylamine (1.12 mmol, 4.5 eq.) was added simultaneously with an ice-cold solution containing tributyl ammonium pyrophosphate (˜0.5 mol) and DMF (1.25 mmol, 5 eq.). The reaction mixture was stirred for an additional 2 hours. Then, 1 mol/L TEAB (2 mL, 8.0 eq.) was added, and the mixture was stirred on ice for 3 hours before the solvent was evaporated. Finally, the product was purified using a Sephadex column, with elution using a 1 mol/L ammonium bicarbonate gradient. The synthesized 5F-UTP was verified by nuclear magnetic resonance spectroscopy and mass spectrometry.

Unmodified ssRNA origami was synthesized following established protocols, and modified RNA origami was generated by incorporating nucleoside analogs. First, DNA plasmids containing T7 promoter and origami sequences were linearized by BamHI-HF restriction enzyme (New England Biolabs) to generate DNA template. Subsequently, the ssRNA was transcribed through HiScribe T7 High Yield RNA Synthesis Kit (New England Biolabs) with 1 μg DNA template per reaction and further purified via Monarch RNA Cleanup Kit (New England Biolabs), according to the manufacturer's protocols. Modified nucleotide triphosphates (m6ATP, m5CTP, Pseudo-UTP, s2UTP, and 5FUTP, all 100 mM in Tris-HCl buffer) and Cy5-UTP (10 mM in Tris-HCl buffer) incorporated into the transcription process to replace the non-modified nucleotide triphosphates at specified ratios. For dFdCTP, the Y839F mutant of T7 polymerase was used to improve the transcription yield. Subsequently, all the transcribed ssRNA was annealed in 1×PBS, with the temperature gradually decreased from 65 to 15° C. at a rate of −1° C. per 30 minutes, facilitating the self-assembly of either regular ssRNA origami or nucleotide analog-integrated ssRNA origami. The confirmation of the origami structures was conducted through 1% agarose gel electrophoresis (TAE buffer, 90 V, 45 min) and the atom force microscope (Bruker).

Cell uptake modified ssRNA origamis: In the uptake experiments, tumor cells were exposed to 2 μg/mL Cy5-labeled RNA ligands in a complete growth medium. To inhibit endocytosis, cells were pre-treated with 5 μM prochlorperazine (APExBio) for 30 minutes prior to RNA samples incubation, serving as a negative control. After 6 h of incubation at 37° C., the cells were washed three times with 1×PBS. The uptake of RNA samples was analyzed using flow cytometry and confocal microscopy. Flow cytometry measurements were performed on a Guava easyCyte Flow Cytometer (Merck) after collecting the cells. For confocal microscopy imaging, cells were stained with Hoechst 33342 (Cell Signaling Technologies) following standard protocols, and imaged using a ZEISS LSM 800 with Airyscan 1 microscopy. FlowJo software (v10.6.2) and Fiji software (v2.1.0) packages were used to quantitively analyze the flow cytometry and the confocal microscopy data.

Innate immune activation by epigenetics nucleoside analogs-integrated ssRNA origamis: RAW 264.7 cells were seeded in 6-well plates at a density of 2×105 cells per well. After 8 hours of incubation, the cells were treated with 1 μg/mL of ssRNA origamis integrated with epigenetic nucleoside analogs. For RT-qPCR analysis, the macrophages were harvested 16 hours after incubation, and the total RNA was extracted using the RNeasy Plus Kit (QIAGEN). Reverse transcription was performed using the NovoScript® Plus All-in-one 1st Strand cDNA Synthesis SuperMix (Novoprotein) to obtain cDNA. The resulting cDNA was used for the real-time PCR performed with NovoStart® SYBR Green Color qPCR SuperMix (Novoprotein) and analyzed with CFX Opus 96 Real-Time PCR System (Bio-Rad). The housekeeping gene mGapdh was used as the endogenous reference. For flow cytometry, the macrophages were harvested 24 hours after incubation, stained with anti-CD 40 FITC and anti-CD 80 PE Cy7 antibodies, and analyzed through a Guava easyCyte Flow Cytometer. FlowJo software (v10.6.2) was used to quantitively analyze the flow cytometry data.

Cytokine analysis in mice by ssRNA origamis: C57BL/6 mice were intraperitoneally administrated with PBS, 150 μg non-modified ssRNA origamis, 150 μg m5C-integrated, or m5C/PseudoU-integrated ssRNA origamis. The serum samples were collected 3 h after the treatment, and then examined for a panel of proinflammatory cytokines, using the Luminex bead-based mouse immune-response panel array, including IFN-γ, TNF-α, IL-1β, and IL-6. The analysis was conducted following the manufacturer's instructions, including cytokine staining, flow cytometry analysis, and data acquisition.

Drug release of FUssROG: To assess stability and drug release, 1 μg of FUssRNA or FUssROG was supplemented with 10% fetal bovine serum in 10 μL of 1×PBS or with 10 μg/mL RNase T2 in 10 mM MES buffer. The mixtures were incubated at 37° C. for different time intervals. Subsequently, 1% agarose gel electrophoresis was performed, and the resulting gel was analyzed using Fiji software (v2.1.0) packages for quantitative analysis of the electrophoresis data.

Tumor cell lines stimulation by FUssROG: Tumor cells were cultured in 6-well plates and treated with PBS, FU, ssRNA origami, and FUssROG under different conditions. RT-qPCR was used to reveal the tumor cell transcripts after FUssROG treatment. Total RNA was extracted from the cells and reverse-transcribed into cDNA. The cDNA samples were then analyzed via the CFX Opus 96 Real-Time PCR System (Bio-Rad) with NovoStart® SYBR Green Color qPCR SuperMix (Novoprotein). The housekeeping gene GAPDH was used as an endogenous reference. Tumor cell-secreted IFN α was measured via enzyme-linked immunosorbent assay (ELISA). Cells were treated with 10 μg/mL 50%- or 100%-integrated FUssROG or an equivalent concentration of FU. After 24 hours of drug treatment, the cell supernatant was collected and the IFNα was quantified through Human IFN α ELISA kit (Absin) following the manufacturer's instructions.

Cell viability assay: The viability of tumor cell lines was assessed after incubation with FU, ssRNA origami, or FUssROG at equivalent concentrations of FU (7.5 μM for 100% integration and 3.75 μM for 50% integration) or ssRNA origami (10 μg/mL) for 48 hours using the CellTiter-Glo Luminescent Cell Viability Assay (Promega) following the manufacturer's protocol. To determine the synergistic effect, the coefficient of drug interaction (CDI) was calculated as the following equation: CDI=AB/(A+B), where AB represents the ratio of the combination group (FUssROG) to the control group (PBS) and A or B represents the ratio of the single drug group (ssRNA origami or FU) to the control group. A CDI value less than 1 indicates synergism, with CDI values less than 0.7 indicating a significant synergistic effect. A CDI value of 1 indicates additivity, while CDI values greater than 1 indicate antagonism.

### EMBODIMENTS

The following embodiments are intended to be illustrative only and not to be limiting in any way.

Embodiment 1: An RNA nanostructure comprising a single RNA strand that includes a plurality of modified nucleotides, wherein the RNA strand is configured to self-assemble into the RNA nanostructure.

Embodiment 2: The nanostructure of claim 1, wherein the single strand of RNA forms parallel crossover cohesion interactions to create the RNA nanostructure.

Embodiment 3: An RNA nanostructure comprising a single RNA strand that includes a plurality of modified nucleotides, wherein the single RNA strand forms parallel crossover cohesion interactions to create the RNA nanostructure.

Embodiment 4: The nanostructure of any one of embodiments 1-3, wherein the modified nucleotides comprise at least one of modified uridine triphosphate (UTPs), modified cytidine triphosphates (CTPs), modified adenosine triphosphates (ATPs), or a combination thereof.

Embodiment 5: The nanostructure of claim 4, wherein the modified ATPs comprise N6-methyladenosine (m6A).

Embodiment 6: An RNA nanostructure comprising a single RNA strand that includes a plurality of modified UTPs and modified CTPs, wherein the RNA strand is configured to self-assemble into the RNA nanostructure.

Embodiment 7: The nanostructure of embodiment 6, wherein the single strand of RNA forms parallel crossover cohesion interactions to create the RNA nanostructure

Embodiment 8: The nanostructure of any one of embodiments 4-7, wherein the modified UTPs comprise pseudouridine (ψ), N1-methylpseudouridine (m1ψ), 2-thiouridine (s2U), 5-Methyluridine, or fluorouracil (5FU).

Embodiment 9: The nanostructure of any one of embodiments 4-7, wherein the wherein the modified CTPs comprise 5-methycytosine (m5C), 5-Hydroxymethylcytidine, or 2′,2′-diflurodeoxycytidine (dFdC).

Embodiment 10: The nanostructure of any one of embodiments 1-9, wherein the RNA is selected from the group consisting of messenger RNA (mRNA), transfer RNA (tRNA), ribosomal RNA (rRNA), small interfering RNA (siRNA), microRNA (miRNA), and synthetic or engineered RNA analogs thereof.

Embodiment 11: The nanostructure of any one of embodiments 1-10, wherein the RNA nanostructure comprises one or more strands of RNA.

Embodiment 12: The nanostructure of any one of embodiments 1-11, wherein RNA nanostructures is formed as a domain of a larger RNA.

Embodiment 13: The nanostructure of any one of embodiments 1-12, wherein the RNA nanostructure is rectangular in shape.

Embodiment 14: The nanostructure of any one of embodiments 1-13, wherein the RNA nanostructure reduces innate immunogenicity.

Embodiment 15: The nanostructure of embodiment 14, wherein the innate immunogenicity is reduced by decreasing a response of a TLR signaling pathway or a response of an RIG-I signaling pathway.

Embodiment 16: A method of reducing innate immunogenicity in a cell, the method comprising contacting the cell with the RNA nanostructure according to any one of embodiment 1-15.

Embodiment 17: The method of embodiment 16, wherein contacting the cell comprises transfecting the cell with the RNA nanostructure or direct incubation of the cells with the RNA nanostructure.

Embodiment 18: A method of reducing innate immunogenicity in a subject in need thereof, the method comprising: administering the RNA nanostructure according to any one of embodiment 1-15.

Embodiment 19: A method of reducing innate immunogenicity in a cell, the method comprising contacting the cell with an RNA nanostructure comprising a single RNA strand that includes a plurality of modified nucleotides, wherein the RNA strand is configured to self-assemble into the RNA nanostructure.

Embodiment 20: The method of embodiment 19, wherein contacting the cell comprises transfecting the cell with the RNA nanostructure or direct incubation of the cells with the RNA nanostructure.

Embodiment 21: A method of reducing innate immunogenicity in a subject in need thereof, the method comprising administering an RNA nanostructure to the subject, wherein the RNA nanostructure comprises a single RNA strand that includes a plurality of modified nucleotides, wherein the RNA strand is configured to self-assemble into the RNA nanostructure.

Embodiment 22: The method of any one of embodiments 19-21, wherein the single strand of RNA forms parallel crossover cohesion interactions to create the RNA nanostructure.

Embodiment 23: The method of any one of embodiments 19-22, wherein the RNA is selected from the group consisting of messenger RNA (mRNA), transfer RNA (tRNA), ribosomal RNA (rRNA), small interfering RNA (siRNA), microRNA (miRNA), and synthetic or engineered RNA analogs thereof

Embodiment 24: The method of any one of embodiments 19-23, wherein the modified nucleotides comprise at least one of modified uridine triphosphate (UTPs), modified cytidine triphosphates (CTPs), modified adenosine triphosphates (ATPs), or a combination thereof.

Embodiment 25: The method of embodiment 24, wherein the modified ATPs comprise N6-methyladenosine (m6A).

Embodiment 26: The method of embodiment 24, wherein the modified UTPs comprise pseudouridine (ψ), N1-methylpseudouridine (m1ψ), 2-thiouridine (s2U), 5-methyluridine, or fluorouracil (5FU).

Embodiment 27: The method of embodiment 24, wherein the wherein the modified CTPs comprise 5-methycytosine (m5C), 5-hydroxymethylcytidine, or 2′,2′-diflurodeoxycytidine (dFdC).

Embodiment 28: The method any one of embodiment 19-27, wherein the innate immunogenicity is reduced by decreasing a response of a TLR signaling pathway or a response of an RIG-I signaling pathway.

Embodiment 29: A method of treating cancer in a subject in need thereof, the method comprising administering an RNA nanostructure to the subject; wherein the RNA nanostructure comprises a single RNA strand that includes a plurality of modified nucleotides, wherein the RNA strand is configured to self-assemble into the RNA nanostructure.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of,” and as such the written description requirement for claiming one or more embodiments of the present invention using the phrase “consisting essentially of” or “consisting of” is met.

What is claimed is:

1. An RNA nanostructure comprising a single RNA strand that includes a plurality of modified nucleotides, wherein the RNA strand is configured to self-assemble into the RNA nanostructure.

2. The nanostructure of claim 1, wherein the RNA is selected from the group consisting of messenger RNA (mRNA), transfer RNA (tRNA), ribosomal RNA (rRNA), small interfering RNA (siRNA), microRNA (miRNA), and synthetic or engineered RNA analogs thereof.

3. The nanostructure of claim 1, wherein the single strand of RNA forms parallel crossover cohesion interactions to create the RNA nanostructure.

4. The nanostructure of claim 1, wherein the modified nucleotides comprise at least one of modified uridine triphosphate (UTPs), modified cytidine triphosphates (CTPs), modified adenosine triphosphates (ATPs), or a combination thereof.

5. The nanostructure of claim 4, wherein the modified ATPs comprise N6-methyladenosine (m6A).

6. The nanostructure of claim 4, wherein the modified UTPs comprise pseudouridine (Y), N1-methylpseudouridine (m1ψ), 2-thiouridine (s2U), 5-methyluridine, or fluorouracil (5FU).

7. The nanostructure of claim 4, wherein the wherein the modified CTPs comprise 5-methycytosine (m5C), 5-hydroxymethylcytidine, or 2′,2′-diflurodeoxycytidine (dFdC).

8. The nanostructure of claim 1, wherein the RNA nanostructure comprises one or more strands of RNA.

9. The nanostructure of claim 1, wherein RNA nanostructures is formed as a domain of a larger RNA.

10. The nanostructure of claim 1, wherein the RNA nanostructure is rectangular in shape.

11. The nanostructure of claim 1, wherein the RNA nanostructure reduces innate immunogenicity, wherein the innate immunogenicity is reduced by decreasing a response of a TLR signaling pathway or a response of an RIG-I signaling pathway.

12. A method of reducing innate immunogenicity in a cell, the method comprising contacting the cell with an RNA nanostructure comprising a single RNA strand that includes a plurality of modified nucleotides, wherein the RNA strand is configured to self-assemble into the RNA nanostructure.

13. The method of claim 12, wherein contacting the cell comprises transfecting the cell with the RNA nanostructure or direct incubation of the cells with the RNA nanostructure.

14. The method of claim 12, wherein the modified nucleotides comprise at least one of modified uridine triphosphate (UTPs), modified cytidine triphosphates (CTPs), modified adenosine triphosphates (ATPs), or a combination thereof.

15. The method of claim 14, wherein the modified ATPs comprise N6-methyladenosine (m6A); wherein the modified UTPs comprise pseudouridine (ψ), N1-methylpseudouridine (m1ψ), 2-thiouridine (s2U), 5-methyluridine, or fluorouracil (5FU); and wherein the wherein the modified CTPs comprise 5-methycytosine (m5C), 5-hydroxymethylcytidine, or 2′,2′-diflurodeoxycytidine (dFdC).

16. The method of claim 12, wherein the innate immunogenicity is reduced by decreasing a response of a TLR signaling pathway or a response of an RIG-I signaling pathway.

17. A method of reducing innate immunogenicity in a subject in need thereof, the method comprising: administering an RNA nanostructure comprising a single RNA strand that includes a plurality of modified nucleotides, wherein the RNA strand is configured to self-assemble into the RNA nanostructure.

18. The method of claim 17, wherein the modified nucleotides comprise at least one of modified uridine triphosphate (UTPs), modified cytidine triphosphates (CTPs), modified adenosine triphosphates (ATPs), or a combination thereof.

19. The method of claim 18, wherein the modified ATPs comprise N6-methyladenosine (m6A), wherein the modified UTPs comprise pseudouridine (ψ), N1-methylpseudouridine (m1ψ), 2-thiouridine (s2U), 5-Methyluridine, or fluorouracil (5FU), and wherein the wherein the modified CTPs comprise 5-methycytosine (m5C), 5-Hydroxymethylcytidine, or 2′,2′-diflurodeoxycytidine (dFdC).

20. The method of claim 17, wherein the innate immunogenicity is reduced by decreasing a response of a TLR signaling pathway or a response of an RIG-I signaling pathway.
